(19) United States
(12) Patent Application Publication
(10) Pub. No.: US 2024/0065829 A1
(43) Pub. Date: Feb. 29, 2024

(54) AURICULAR RECONSTRUCTION USING 3D PRINTED AUTOLOGOUS CARTILAGE TISSUE

(71) Applicants: Technion Research & Development Foundation Limited, Haifa (IL); Tel HaShomer Medical Research Infrastructure and Services Ltd., Ramat-Gan (IL)

(72) Inventors: Shulamit LEVENBERG, Haifa (IL); Shira LANDAU, Haifa (IL); Shay DUVDEVANI, Ramat Gan (IL)

(73) Assignees: Technion Research & Development Foundation Limited, Haifa (IL); Tel HaShomer Medical Research Infrastructure and Services Ltd., Ramat-Gan (IL)

(21) Appl. No.: 18/270,852

(22) PCT Filed: Jan. 4, 2022
(86) PCT No.: PCT/IL2022/050016

§ 371 Date: Jul. 4, 2023

Related U.S. Application Data
(60) Provisional application No. 63/133,453, filed on Jan. 4, 2021.

(51) Int. Cl. A61F 2/18; A61L 27/38

(52) U.S. Cl. CPC A61F 2/18; A61L 27/3817; A61F 2002/183; A61L 2430/06

(57) ABSTRACT

An aspect of some embodiments of the invention relates to methods of manufacturing a 3D printed implant, comprising printing a mold of a scaffold, generating said scaffold from said mold, seeding cells on said scaffold, where said generating said scaffold comprises generating parts of said scaffold with different levels of stiffness.

### RELATED APPLICATION/S

This application claims the benefit of priority of U.S. Provisional Patent Application No. 63/133,453 filed on 4 Jan. 2021, the contents of which are incorporated herein by reference in their entirety.

### FIELD AND BACKGROUND OF THE INVENTION

The present invention, in some embodiments thereof, relates to 3D printing of tissues and, more particularly, but not exclusively, to auricular reconstruction using 3D printed autologous cartilage tissue. Microtia is a congenital auricle malformation that affects one in every 5,000 to 7,000 births worldwide. The malformation of the auricle can impact the physical and mental wellbeing of the child. The auricle is an identifying feature of a face, and an absence or deformity can have a strong impact on the child's self-image and sense of self-worth. Current solutions for this problem is the use of one of the techniques for autogenous costal cartilage described by Brant and Nagata or artificial plastic implant like “Medpor” which is used as material for the fabrication of a 3D framework.

Additional background art includes:

Baluch N., et al, “Auricular reconstruction for microtia: a review of available methods”. Disclosing a review of available auricular reconstruction methods.

Cao Y. et al, “Transplantation of chondrocytes utilizing a polymer-cell construct to produce tissue-engineered cartilage in the shape of a human ear”. Disclosing the feasibility of growing tissue-engineered cartilage in the shape of human ear using chondrocytes.

Bichara D. A., et al, “The tissue-engineered auricle: Past, present, and future”. Disclosing a review of reconstruction, repair, and regeneration of the external auricular tissue.

Cohen B. P., et al, “Tissue engineering the human auricle by auricular chondrocyte-mesenchymal stem cell co-implantation”. Disclosing studies that demonstrate successful engineering of a patient-specific human auricle using exclusively human cell sources without extensive in vitro tissue culture prior to implantation.

Reiffel A. J., et al, “High-Fidelity Tissue Engineering of Patient-Specific Auricles for Reconstruction of Pediatric Microtia and Other Auricular Deformities”. Disclosing high-fidelity, biocompatible, patient-specific tissue-engineered constructs for auricular reconstruction which largely mimic the native auricle both biomechanically and histologically, even after an extended

Zhou G., et al, “In Vitro Regeneration of Patient-specific Ear-shaped Cartilage and Its First Clinical Application for Auricular Reconstruction”. Disclosing an engineered patient-specific ear-shaped cartilage in vitro. The cartilage was used for auricle reconstruction of five microtia patients and achieved satisfactory aesthetical outcome with mature cartilage formation during 2.5 years follow-up in the first conducted case.

Martínez Ávila H., et al, “3D bioprinting of human chondrocyte-laden nanocellulose hydrogels for patient-specific auricular cartilage regeneration”. Disclosing that NFC-A bioink supports redifferentiation of hNCs while offering proper printability in a biologically relevant aqueous 3D environment.

Jiang R., et al, “Three-dimensional bioprinting of auricular cartilage: A review”. Disclosing a review in 3D bioprinting.

Chen Y., et al, “Noninvasive in vivo 3D bioprinting”. Disclosing a digital near-infrared (NIR) photopolymerization (DNP)-based 3D printing technology that enables the noninvasive in vivo 3D.

Kang H. W., et al, “3D bioprinting system to produce human-scale tissue constructs with structural integrity”. Disclosing capabilities of an integrated tissue-organ printer (ITOP) by fabricating mandible and calvarial bone, cartilage and skeletal muscle.

Kim B. S., et al, “Decellularized Extracellular Matrix-based Bioinks for Engineering Tissue- and Organ-specific Microenvironments”. Disclosing a review to discuss a new paradigm of dECM-based bioinks able to recapitulate the inherent microenvironmental niche in 3D cell-printed constructs.

Suhun C., et al, “3D cell-printing of biocompatible and functional meniscus constructs using meniscus-derived bioink”. Disclosing three-dimensional (3D) cell-printed meniscus constructs using a mixture of polyurethane and polycaprolactone polymers and cell-laden decellularized meniscal extracellular matrix (me-dECM) bioink with high controllability and durable architectural integrity.

Kaplan B., et al, “Rapid prototyping fabrication of soft and oriented polyester scaffolds for axonal guidance”. Disclosing a fabrication procedure based on 3D printing to generate highly ordered and anatomically personalized, polyester scaffolds for soft tissue regeneration.

### SUMMARY OF THE INVENTION

According to an aspect of some embodiments of the present invention there is provided a medical grade implant, comprising:
a. a biodegradable scaffold;
b. a plurality of seeded cells on the surface of said scaffold;
wherein said biodegradable scaffold comprises parts with different levels of stiffness.

According to some embodiments of the invention, the parts with different levels of stiffness comprise different quantities of scaffold material.

According to some embodiments of the invention, parts having high levels of stiffness comprise more scaffold material.

According to some embodiments of the invention, parts having low levels of stiffness comprise less scaffold material.

According to some embodiments of the invention, parts having low levels of stiffness comprise openings.

According to some embodiments of the invention, parts having high levels of stiffness comprise less openings or no openings at all.

According to some embodiments of the invention, low levels of stiffness are from about 1.5 MPa to about 5 MPa.

According to some embodiments of the invention, low levels of stiffness are about 2 MPa.

According to some embodiments of the invention, high levels of stiffness are from about 15 MPa to about 30 MPa.

According to some embodiments of the invention, high levels of stiffness are about 19 MPa.

According to some embodiments of the invention, the scaffold comprises an ultimate tensile strength of from about 10% PCL/0.25 MPa to 30 30% PCL/2 MPa.

According to some embodiments of the invention, parts that comprise more scaffold material comprise from about 5% to about 80% more scaffold material than parts comprising less scaffold material.

According to some embodiments of the invention, parts comprising higher levels of stiffness provide stability of said implant after implantation.

According to some embodiments of the invention, the implant allows cell growth and regeneration of cartilage tissue at the implant site.

According to an aspect of some embodiments of the present invention there is provided a method of manufacturing a 3D printed implant, comprising:
a. printing a mold of a scaffold;
b. generating said scaffold from said mold;
c. seeding cells on said scaffold;
wherein said generating said scaffold comprises generating parts of said scaffold with different levels of stiffness.

According to some embodiments of the invention, the method further comprises virtually planning the scaffold using a scanned image of an anatomical structure of a subject in need of said 3D printed implant.

According to some embodiments of the invention, the method further comprises virtually generating a mold for the planned scaffold to be used as instructions for the printing.

According to some embodiments of the invention, the printing comprises using at least one printing material for the printing.

According to some embodiments of the invention, generating parts with different levels of stiffness comprises printing parts of the mold with more of the at least one printing material than other parts in the mold.

According to some embodiments of the invention, parts comprising more of the at least one printing material in the mold provide stiffer parts in the scaffold during the generating.

According to some embodiments of the invention, parts comprising less of the at least one printing material comprise openings.

According to some embodiments of the invention, in the parts comprising openings less scaffold material is retained during the generating of the scaffold from the mold.

According to some embodiments of the invention, less retained scaffold material provides parts with lower levels of stiffness in the scaffold.

According to some embodiments of the invention, parts comprising more printing material comprise less openings or no openings at all.

According to some embodiments of the invention, in the parts comprising less openings or no openings at all more scaffold material is retained during the generating of the scaffold from the mold.

According to some embodiments of the invention, more retained scaffold material provides parts with higher levels of stiffness in the scaffold.

According to some embodiments of the invention, lower levels of stiffness are from about 1.5 MPa to about 5 MPa.

According to some embodiments of the invention, lower levels of stiffness are about 2 MPa.

According to some embodiments of the invention, higher levels of stiffness are from about 15 MPa to about 30 MPa.

According to some embodiments of the invention, higher levels of stiffness are about 19 MPa.

According to some embodiments of the invention, the scaffold comprises an ultimate tensile strength of from about 10% polycaprolactone (PCL)/0.25 MPa to about 30% PCL/2 MPa.

According to some embodiments of the invention, the parts that comprise more scaffold material comprise from about 5% to about 80% more scaffold material than parts comprising less scaffold material.

According to some embodiments of the invention, parts comprising higher levels of stiffness provide stability of the implant after implantation.

According to some embodiments of the invention, the implant allows cell growth and regeneration of cartilage tissue at the implant site.

According to an aspect of some embodiments of the present invention there is provided a method for producing a custom implant for cartilage repair, comprising:
a. manufacturing a three-dimensional mesh mold using a scanned image of an anatomical structure of a subject in need; wherein the manufactured three-dimensional mesh mold serves as a supporting scaffold for the custom implant;
b. generating said scaffold from the mold;
c. seeding cells on the scaffold;
wherein the generating the scaffold comprises generating reinforcement zones in the scaffold at predefined areas that allow the structural stability of the implant after implantation; further wherein the implant allows cell growth and the regeneration of cartilage tissue at the implant site.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and/or images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of an exemplary manufacturing method of the invention, according to some embodiments of the invention;

FIGS. 2a-2i are images of an exemplary scaffold preparation, according to some embodiments of the invention;

FIG. 2j is a flowchart of an exemplary method of scaffold preparation, according to some embodiments of the invention;

FIG. 3 is a schematic illustration of a Polycaprolactone (PCL)-Auricle scaffold fabrication process, according to some embodiments of the invention;

FIGS. 4a-4e are images of the ear-shape scaffold characterization, according to some embodiments of the invention;

FIGS. 5a-5b are images of the isolated chondrocytes and MSCs morphology and characterization, according to some embodiments of the invention;

FIGS. 6a-6c are images of the characterization of in vitro scaffold-free chondrogenic differentiation potential, according to some embodiments of the invention;

FIGS. 7a-7b are images of the characterization of in vitro scaffold-base chondrogenic differentiation potential, according to some embodiments of the invention;

FIGS. 8a-8c are images of the in vivo implantation of human auricle grafts in murine model, according to some embodiments of the invention;

FIG. 9 is a schematic representation of an exemplary experimental design to prove the feasibility of the invention, according to some embodiments of the invention;

FIG. 10 is a schematic representation of an exemplary method of preparation of disc scaffold, according to some embodiments of the invention;

FIGS. 11a-c are images of the characterization of the microtia auricular chondrocytes (mAC), costal chondrocytes (CCs), and adipose-derived MSCs, according to some embodiments of the invention;

FIGS. 12a-b are images of the characterization of the in vitro chondrogenic potential of chondrocytes and MSCs, according to some embodiments of the invention;

FIG. 13 are images and graph of the characterization of in vitro chondrogenic differentiation potential of scaffold-free MSCs, according to some embodiments of the invention;

FIGS. 14a-b are images of exemplary scaffolds without cells, according to some embodiments of the invention.

FIGS. 14c-e are images of exemplary cartilage formation within constructs of different cell combinations, according to some embodiments of the invention;

FIGS. 15a-d are images of exemplary cartilage formation within constructs cultured for 10 days versus 6 weeks in vitro, according to some embodiments of the invention;

FIG. 16 is an image of an exemplary confocal microscopy images of a full-sized auricle seeded with GFP-labeled cells, according to some embodiments of the invention; and

FIG. 17 is an exemplary stress-strain curve of a 12-week implant, according to some embodiments of the invention.

### DESCRIPTION OF SPECIFIC EMBODIMENTS OF THE INVENTION

The present invention, in some embodiments thereof, relates to 3D printing of tissues and, more particularly, but not exclusively, to auricular reconstruction using 3D printed autologous cartilage tissue.

### Overview

An aspect of some embodiments of the invention relates to 3D printed implants comprising autologous tissues. In some embodiments, the implant comprises a scaffold having different zones, each zone comprising a different value of stiffness. In some embodiments, the stiffer zones provide physical stability to the form of the implant. In some embodiments, the scaffold is made of medical grade biodegradable material which is primarily covered with autologous cells, then over time, while the scaffold degrades, autologous tissue will take the place and function of the scaffold.

An aspect of some embodiments of the invention relates to methods of manufacture of biodegradable implants. In some embodiments, a mold of a scaffold is 3D-printed using a computerized design. In some embodiments, the mold comprises zones where more scaffold material will be retained and zones where less scaffold material will be retained. In some embodiments, the zones where more scaffold material will be retained are zones without openings in the mold. In some embodiments, the zones where more scaffold material will be retained are zones having higher levels of stiffness in relation to the zones where less scaffold material will be retained. In some embodiments, the zones where less scaffold material will be retained comprise openings in the mold. In some embodiments, the zones where less scaffold material will be retained are zones having lower levels of stiffness in relation to the zones where more scaffold material will be retained. In some embodiments, the mold is then melted away from the scaffold, the scaffold is optionally cut to refine its form and cells are then seeded on the scaffold for later implantation in a subject in need thereof. In some embodiments, the scaffold is a biodegradable scaffold that, after implantation, over time is degraded leaving only the cells. In some embodiments, zones having more scaffold and/or zones having less scaffold are strategically chosen according to the required reinforcements areas of the implant to maintain a complex topography of the outer ear and additionally to provide micropores to allow cell adhesion for the effective production of stable cartilage.

An aspect of some embodiment of the invention relates to an efficient method to bioengineer a full-scale human autologous auricle. In some embodiments, a CT scan-based auricle construct is comprised of a 3D-printed clinical-grade biodegradable PCL scaffold loaded with patient-derived chondrocytes produced from either auricular cartilage or costal cartilage biopsies combined with adipose-derived MSCs and auricular perichondrium cells. In some embodiments, in addition, the auricle construct comprises strategically reinforced regions to ensure the mechanical stability necessary to withstand the surrounding stress upon implantation. In some embodiments, a suitable material for the auricle construct is chosen to potentially allow a correct and better cartilage formation and graft integration. In some embodiments, to increase cartilage formation, decellularized extracellular matrix bioinks are used. In some embodiments, one medical-grade material is used at varying densities, which is achieved by 3D-printing molding. In some embodiments, the construct comprises predetermined reinforced regions that maintain its structure upon implantation. In some embodiments, a potential advantage of the invention is that it potentially allows for a non-complex production process and potentially enables rapid production of a large mass of constructs with a fine structure and pores appropriate for cell attachment. Another potential advantage of the invention is that it potentially provides a fast and simple method for engineering a medical-grade auricle. In some embodiments, the use of 3D-printing molding with tissue-specific reinforced regions, along with a short in vitro culture period of scaffolds with auricular cartilage cells, potentially yields patient-specific ear-shaped cartilage, suitable for transplants for microtia patients. In some embodiments, in cases of a complete lack of auricular cartilage, such as anotia and trauma, the method can potentially be modified to integrate costal cartilage cells. In some embodiments, this invention can potentially be expanded into more products in the clinic, such as nasal reconstruction or other orthopedic implants.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

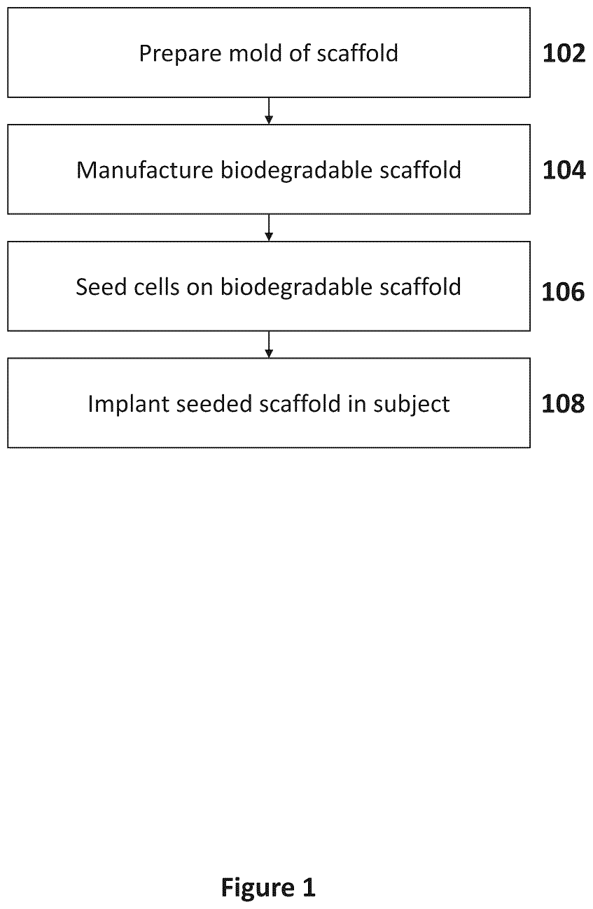

Referring now to the drawings, FIG. 1 illustrates a principal of some embodiments of the invention. In some embodiments, the principal of the invention relates to the preparation of implantable cartilaginous scaffolds. In some embodiments, in general, the manufacturing method comprises one or more of: the preparation of a mold of a scaffold 102, the manufacture of the scaffold according to the mold 104, seeding of cells on the scaffold 106 and implantation of the seeded scaffold in the subject 108, as will be further explained in the exemplary method of manufacture below.

In some embodiments, an exemplary method of preparation of the mold of the scaffold includes the design of the required implant in negative form in a dedicated software. In some embodiments, the mold of the scaffold comprises zones with different types and quantities of openings. In some embodiments, openings refer to zones in the mold where substances can pass through, for example zones where the material of which the scaffold is made can pass through and/or occupy during the process of making the scaffold. In some embodiments, the types and quantities of openings directly correlate with the level of strength of the implant at those locations. In some embodiments, when designing the implant, the user identifies zones that require different levels of strength. In some embodiments, for example, in zones where higher levels of strength are required, the scaffold will comprise less or no openings, as will be further explained in the exemplary method of manufacture below.

In some embodiments, the stiffness of the scaffold plays a double role. In some embodiments, in the short term, the stiffness of the scaffold potentially helps during the seeding phase to the better seeding of the cells on the scaffold itself. In some embodiments, in the long term, the stiffness of the scaffold potentially helps during the implantation process in the subject. In some embodiments, the stiffness of the scaffold helps keeping the wanted form of the implant until the scaffold is replaced with cells.

In some embodiments, the scaffold comprises at least two levels of stiffness. In some embodiments, the stiffness of the less stiff (or soft) parts is of from about 1.5 MPa to about 5 MPa, optionally from about 1 MPa to about 7 MPa, optionally from about 0.5 MPa to about 10 MPa, for example 2 MPa, 4 MPa, 6 MPa. In some embodiments, the stiffness of the more stiff (or stiff) parts is of from about 17 MPa to about 20 MPa, optionally from about 15 MPa to about 30 MPa, optionally from about 12 MPa to about 40 MPa, for example 19 MPa, 22 MPa, 25 MPa.

In some embodiments, the scaffold comprises an ultimate tensile strength of from about 10% PCL/0.25 MPa to about 30% PCL/2 MPa.

In some embodiments, the stiff parts comprise more scaffold material than the soft parts. In some embodiments, the stiff parts comprise from about 40% to about 60% more scaffold material than the soft parts. Optionally from about 30% to about 70% more scaffold material. Optionally from about 20% to about 80% more scaffold material. Optionally from about 5% to about 80% more scaffold material. For example, 50%, 35%, 65%, 48%, or higher % or lower % or any interval of % in between. In some embodiments, the optimal percentage of higher quantity of material in the stiff parts is of about 50%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following exemplary manufacturing methods.

### Exemplary Manufacturing Methods

Reference is now made to the following exemplary manufacturing methods, which together with the above descriptions illustrate some embodiments of the invention in a non limiting fashion.

### Exemplary Scaffold Preparation

In some embodiments, solid modeling computer-aided design and computer-aided engineering computer programs such as Solid Works® and 3D Slicer (Prusa) are used to design the molds. In some embodiments, other software programs can be used in similar fashion, and are also included in the scope of some embodiments of the invention. In some embodiments, the mold is designed as a negative auricle (from a CT scan) within a box with inlet channels that allow loading of the PCL solution. In some embodiments, the mold comprises zones comprising infills and zones left as void without infills. In some embodiments, the zones comprising the infill comprised a 50% rectilinear infill. In some embodiments, the zones in the mold where stiffer zones are required were left as a void with no infill.

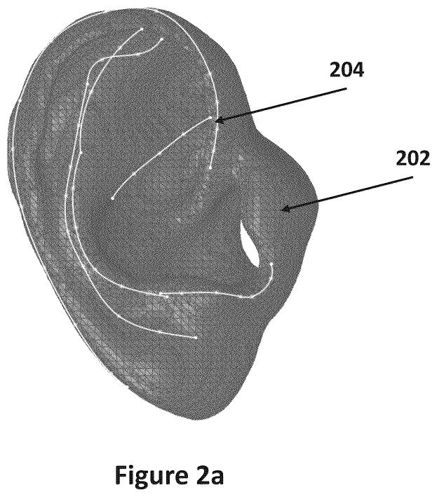

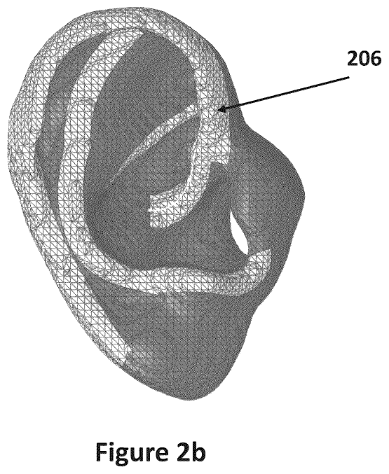

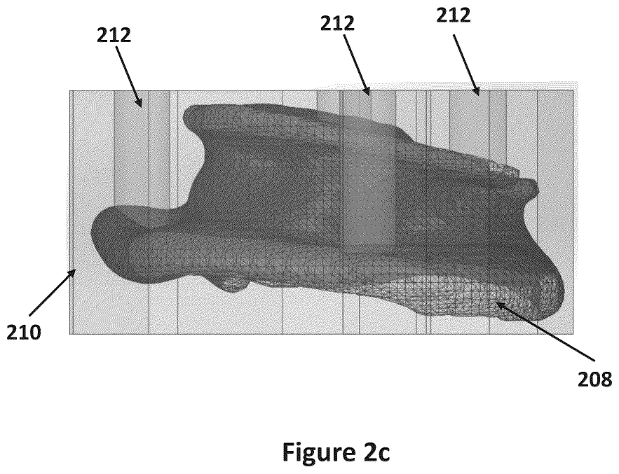

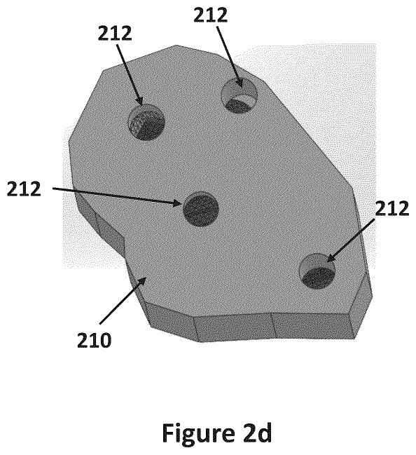

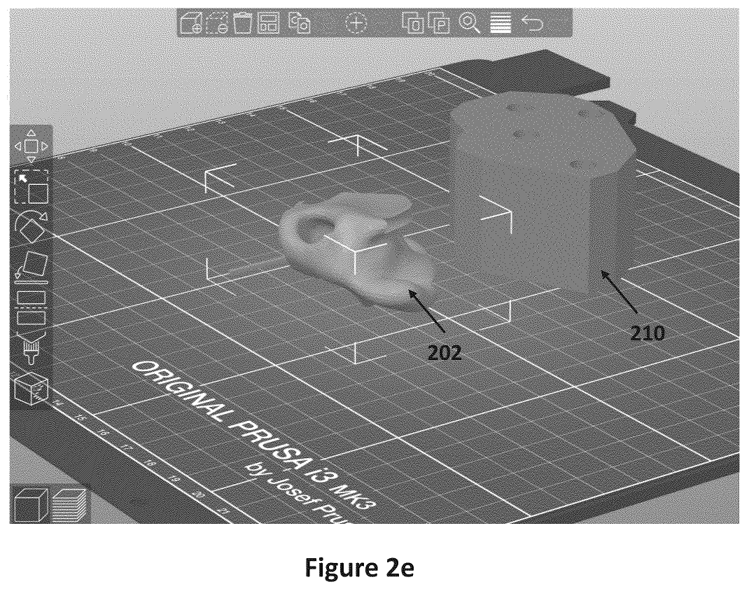

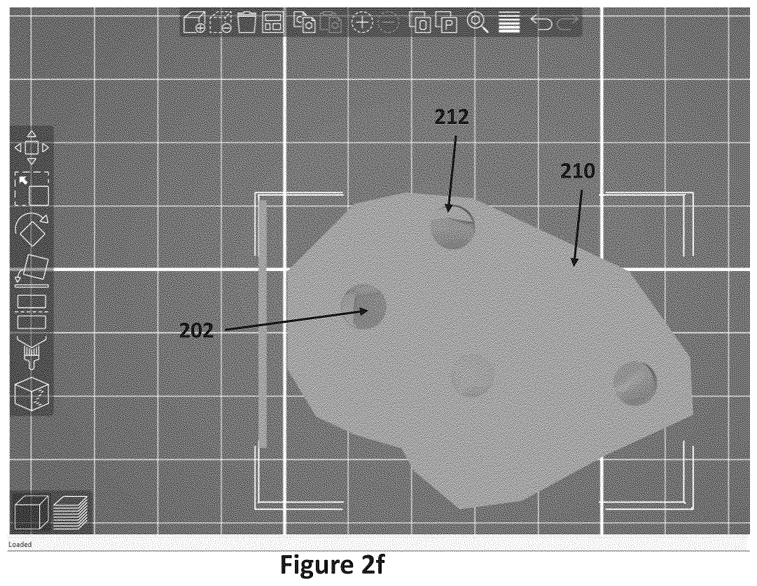

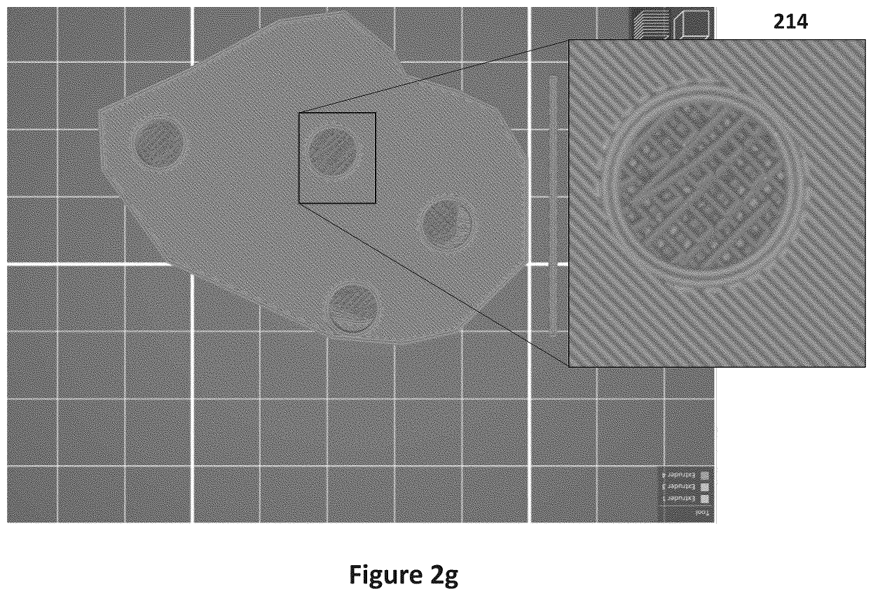

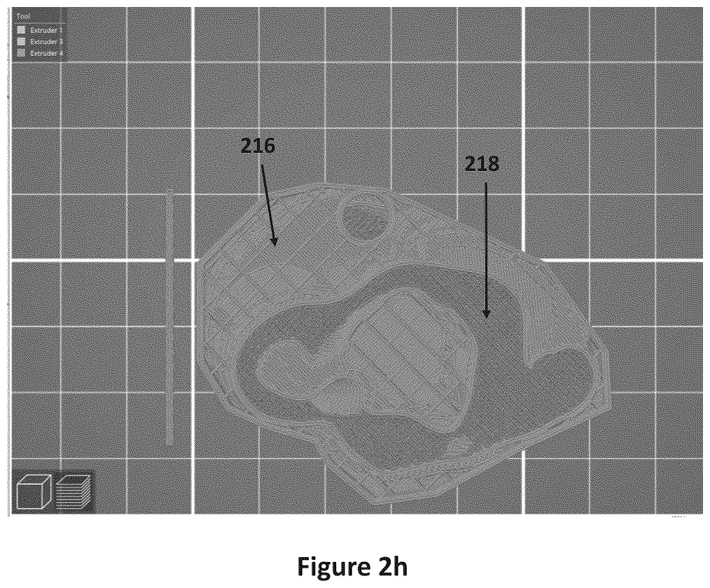

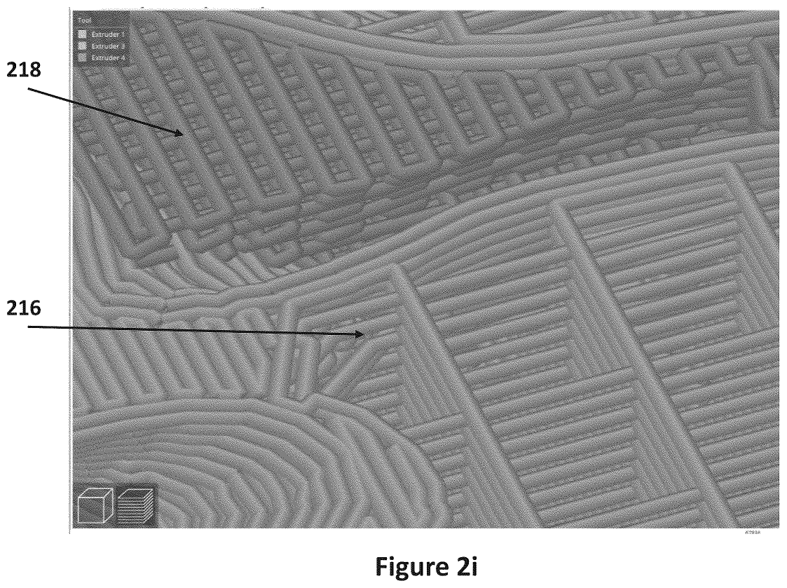

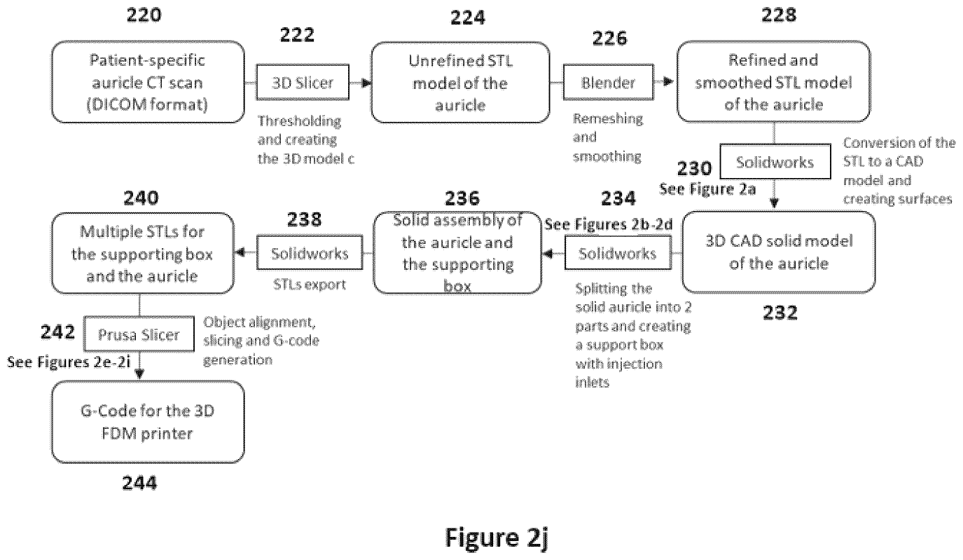

Referring now to FIGS. 2a-2i, showing illustrations of the scaffold preparation, according to some embodiments of the invention. Additionally, referring to FIG. 2j, showing a flowchart of an exemplary method for scaffold preparation, according to some embodiments of the invention. In some embodiments, an example of a scaffold preparation is as follows: a CT scan, for example a DICOM CT scan (220 in FIG. 2j), of the auricle 202 is imported for example into SolidWorks® (230 in FIG. 2j), and then desired stiffer areas are manually sketched 204 on the object (FIG. 2a). In some embodiments, prior to importing into SolidWorks®, an unrefined STL file is processed (222-224 in FIG. 2j), for example, in Blender (open-source 3D creation suite) (226 in FIG. 2j) to re-mesh and smooth the object (228 in FIG. 2j). In some embodiments, the previously sketched areas are then broadened 206 to the desired thickness (FIG. 2b). In some embodiments, the stiffer areas are subtracted 208 from the auricle and the object is positioned in a virtual box 210 (FIG. 2c) (236 in FIG. 2j). In some embodiments, openings (Hollows) 212 are added to the box to enable polymer solution loading (FIGS. 2c and 2d) (see also 232-238 in FIG. 2j).

In some embodiments, the information about the box with the subtracted auricle 210 and the auricle with the subtracted stiffer areas 202 is imported, for example, to the SLICER software (Prusa) (FIG. 2e) (242 in FIG. 2j). In some embodiments, in the software, the box with the subtracted auricle 210 and the auricle with the subtracted stiffer areas 202 are aligned (FIG. 2f), and then “slicing” is performed on them (FIG. 2g). View 214 in FIG. 2g shows a zoom in of the object after the “slicing” is performed. In some embodiments, a 10% rectilinear infill 216 is applied to the box and a 50% rectilinear infill 218 is applied to the auricle. FIG. 2h shows a mid-section of what is shown in FIG. 2g, it can be seen that the darker areas 218, which are those of the auricle, are those with 50% rectilinear infill, while the lighter areas 216, which are those of the box, are those with 10% rectilinear infill. FIG. 2i is a zoom in to show the difference between a 10% rectilinear infill 216 and a 50% rectilinear infill 218. In some embodiments, then the file is ready to be provided for printing (244 in FIG. 2j).

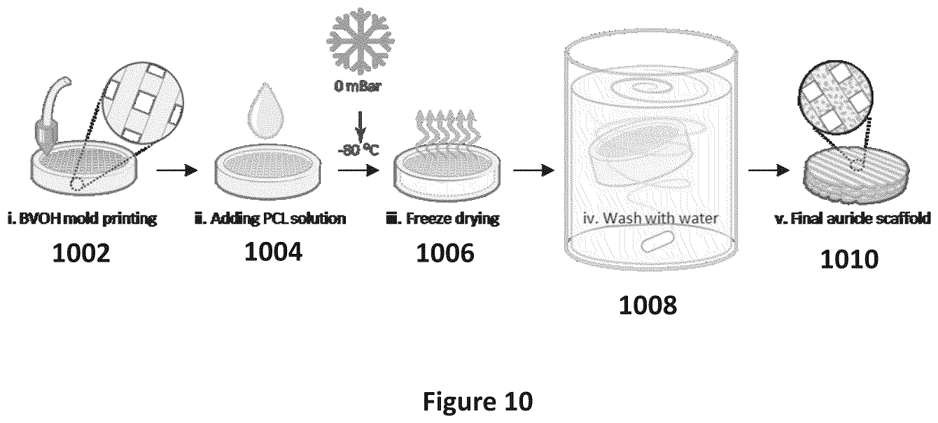

In some embodiments, to create the PCL-discs, 50% infill was designed in a 9 mm diameter discs. In some embodiments, exemplary methods of manufacture of discs are shown below and in FIG. 10.

In some embodiments, the molds are printed out of water-soluble butenediol vinyl alcohol (BVOH) using a Prusa MK2.5 printer with a 0.4 mm nozzle. In some embodiments, other water-solutions materials can also be used.

In some embodiments, to prepare the PCL solution, medical-grade PCL (Poly-Med) is dissolved in dioxane to form a 10% (w/v) solution overnight under shaking and heating to 70° C. In some embodiments, PCL solution is then injected through the inlet channel using a 1 ml pipettor. In some embodiments, the filled molds are then placed in a −80° C. freezer overnight, followed by lyophilization overnight. In some embodiments, to dissolve the BVOH molds, constructs are washed with distilled water overnight (water is optionally replaced every few hours). In some embodiments, the auricles are then separated easily from the polymer that accumulated in the supporting box. In some embodiments, PCL auricles are then sterilized by additional overnight lyophilization and soaking them in 70% ethanol solution for 3 hours.

In some embodiments, to fabricate PCL-discs, a similar process is performed, for example, discs are filled with 600 μl PCL solution and go through the same process as the PCL-auricles. In some embodiments, prior sterilization, the discs are cut into smaller 6 mm discs to ensure the reproducibility of the identical discs.

It should be understood that the methods herein are exemplary methods and that deviations of what is disclosed herein in time, quantities and operations which provide the same or similar results are also included in the scope of some embodiments of the invention.

### Exemplary SEM, Micro CT Imaging and Mechanical Testing

In some embodiments, scaffold morphology is examined using a scanning electron microscope (SEM). In some embodiments, prior scanning, samples are coated with a gold-palladium mixture using a Polaron gold coater and then scanned with a Quanta 200 microscope (FEI). In some embodiments, other methods of examination can also be used.

In some embodiments, stress-strain curves are generated using a rheometer. In some embodiments, samples are imaged after testing to find the cross-section area essential for assessing the stretch, see below.

### Exemplary Methods of Cell Culture

### Exemplary Cell Isolation

In some embodiments, cell types used in the implants can be one or more of microtia auricular chondrocytes (mACs), costal chondrocytes (CCs), microtia auricular adipose-derived mesenchymal stem cells (maMSCs), and costal adipose-derived mesenchymal stem cells (cMSCs).

In some embodiments, cells from patients diagnosed with microtia are used.

In some embodiments, to isolate the chondrocytes, either rib cartilage or auricular cartilage were cut into small pieces, followed by a PBS wash with 1% penicillin/streptomycin (Pen/Strep) (BI), and incubation with collagenase 2 for 12-14 hours. In some embodiments, the tissue solution was then filtered with a 100 μm strainer. In some embodiments, growth media (40 ml of DMEM F12 with 10% FBS and 1% Pen strep) was added to the filtered solution followed by centrifugation of 2000 RPM for 10 minutes. In some embodiments, media was then aspirated and cells were mixed with growth media and seeded in the density of 5000 cells/mm2.

In some embodiments, to isolate adipose-derived mesenchymal stem cells (MSCs), either rib fat or auricular fat were cut into small pieces. 2 mg/ml collagenase 1 (in DMEM) was added to the tissue (10 ml collagenase for every 3 gr of tissue) and incubated for 1 hour at 37° C. DMEM F12 with 10% FBS was then added to the tissue-digested solution (the same amount as the collagenase) followed by double filtration using 100 μm and then 40 μm strainers. In some embodiments, the solution is then centrifuged for 7 minutes at 500 g. In some embodiments, the media is then aspirated and cells are then mixed with growth media (DMEM F12 with 10% FBS and 1% Pen/Strep). In some embodiments, one day later, the media was changed to Nutristem (biological industries). In some embodiments, for both cell types media was exchanged every 2-3 days.

It should be understood that the methods herein are exemplary methods and that deviations of what is disclosed herein in time, quantities, biological materials, chemical compounds and operations which provide the same or similar results are also included in the scope of some embodiments of the invention.

### Cell Seeding

In some embodiments, prior seeding either PCL discs or auricles were washed with PBS for three times and then washed with growth media, to create a hydrophilic environment for the seeding and stain the construct with phenol red color.

In some embodiments, chondrocytes with and without MSCs were trypsinized using 2× trypsin and mixed with 7.5 U Thrombin (EVICEL) that was divided into 8 test tubes. 15 mg/ml BAC2 solution (fibrinogen) (EVICEL the same amount as the Thrombin) was then mixed with the thrombin-cells solution followed by seeding into the auricle or scaffold. In some embodiments, unloaded areas were detected easily as the phenol red color disappeared from the seeded areas. In some embodiments, cells were seeded at a density of 50×10⁶ cells/ml. In some embodiments, for 100% size auricle—800 μl of the fibrin solution was used and for the 70% size auricle—600 μl of the fibrin solution was used accordingly. In some embodiments, for the PCL discs, 20 microliters of fibrin were used.

In some embodiments, seeded constructs were incubated for 1 hour at 37° C. followed by the addition of growth media. In some embodiments, two days following seeding, differentiation medium was added: DMEM F12 (biological industries), 1% Pen/Strep, transforming growth factor β3 (TGF-□3) (10 ng/ml, Prospec), ITS (Corning), L-ascorbic acid 2-phosphate (50 μg/ml, Sigma), Dexamethasone (100 nM, Sigma), Amphotericin B (20 μg/ml).

In some embodiments, the media amount for each construct was: 150 ml for the 100% size auricle, 100 ml for the 70% size auricles, and 4 ml for the discs.

### Chondrocytes Plugs

In some embodiments, different combinations of chondrocytes (400,000 cells, auricular or rib origin) with or without 50% adipose-derived MSCs (auricular or rib origin) were mixed and centrifuged for 5 minutes at 400 g creating plugs. In some embodiments, in the first two days, basal media was added to the plugs and was then changed to the differentiation media. In some embodiments, the media was changed every 2-3 days. In some embodiments, after 6 weeks of culture, plugs were fixed with 4% Paraformaldehyde followed by 3 PBS washes and were incubated in 30% (wt/vol) sucrose solution, embedded in optimal cutting temperature compound (Tissue-Tek), and frozen for subsequent cryosectioning (5-7 μm).

### Grafts Implantation

In some embodiments, athymic nude mice (7 to 9 wk old; Harlan Laboratories) were anesthetized using isofluorane. In some embodiments, a small (for the discs) and a large (for the auricles) incision were made and grafts were placed within it. In some embodiments, the skin was then sutured with 5-0 absorbable sutures. In some embodiments, after 12 weeks, mice were sacrificed, followed by the excision of the grafts. In some embodiments, grafts were then cut into 3 pieces for mechanical testing, staining, RNA analysis, and biochemical analysis.

In some embodiments, for the staining, the samples were fixed in 4% paraformaldehyde.

### Immunofluorescent and Biochemical Staining

In some embodiments, 4% Paraformaldehyde was added to the constructs for 20 min in order to fixate them, followed by permeabilization with 0.3% Triton X-100 (Bio Lab Ltd) for 10 min. In some embodiments, constructs were then washed with PBS and immersed in BSA solution (5%; Millipore) overnight. In some embodiments, samples were then incubated with either anti-human collagen 1 (1:200; Abcam), anti-human collagen 2 (1:200; Abcam, and anti-human aggrecan (1:500; r&d) overnight at 4° C. Constructs were then treated with Cy3-labeled (1:100; Jackson Immunoresearch Laboratory) and/or Alexa-488 (1:400; ThermoFisher Scientific) and Alexa-647 (1:400; ThermoFisher Scientific) secondary antibodies mixed with DAPI (Sigma-Aldrich) for 3 h at room temperature.

In some embodiments, constructs were dehydrated in ethanol and embedded in paraffin at 65° C. Sections were cut at 5 μm thickness, deparaffinized with xylene and rehydrated and then stained with Alcian-blue, safranin-O with fast green with hematoxylin and eosin.

### Biochemical Assays

In some embodiments, in-vitro and in-vivo samples were digested with papain solution, 40 μg/ml diluted in 20 nM ammonium acetate, 1 mM EDTA, and 2 mM dithiothreitol for 48 hours at 65° C. DNA content was measured by hoechst assay. In some embodiments, proteoglycan amount was quantified by measuring the amount of sulfated GAG using the 1,9-dimethylmethylene blue (DMMB) dye binding assay (PMID: 3091074). In some embodiments, collagen content was quantified by adding 6N hydrochloric acid for 18 hours at 110° C., then, hydroxyproline amount was measured by using chloramine-T/Ehrlich's assay (PMID: 13786180).

### Exemplary Results

### Scaffold Fabrication

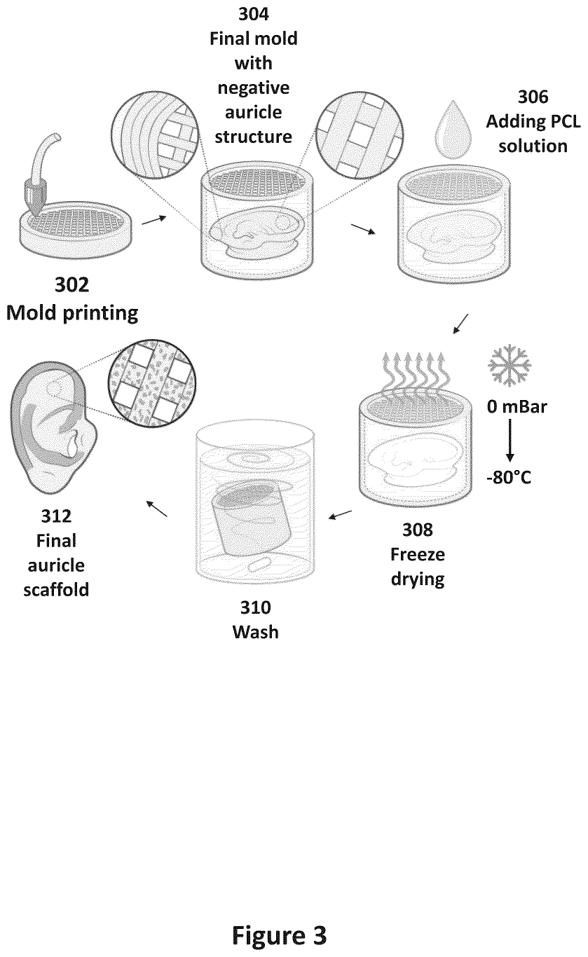

Referring to FIG. 3, showing a schematic representation of an exemplary method of manufacturing a auricle, according to some embodiments of the invention. In some embodiments, the production process of the PCL scaffold is based on a 3D-printing and freeze-drying technique. In some embodiments, to create the 3D printed molds, a STL file of a CT scan of an auricle; the file was edited in Solidworks to lower the resolution of the structure. In some embodiments, next, the file was transformed to the Slicer software and the BVOH meshed molds 304 were printed 302 with or without the denser supporting frames. In some embodiments, then, medical-grade PCL solution 306 was poured into the mold followed by rapid construct freezing to avoid evaporation 308. In some embodiments, lyophilization was then applied to form pores within the polymer bulk, followed by water washes to remove the BVOH mold 310. In some embodiments, the final auricle was then dried, sterilized, and ready for seeding 312.

### Ear-Shape Scaffold Characterization

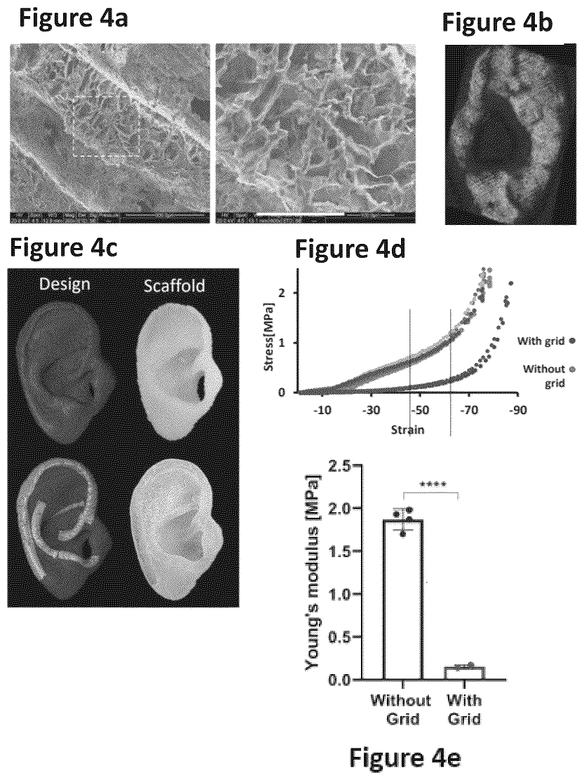

Referring now to FIGS. 4a-e, showing FIG. 4a, SEM images of the structure showing the large pores (large openings) that result from the grid mold and the small pores (small openings) that result from the freeze-drying process, according to some embodiments of the invention. Scale bar=500 μm (left image), 100 μm (right image). The images indicate that the large pore size (result from the pore pattern) is approximately 500 μm, while the small pore size ranges from 10-100 μm. FIG. 4b, confocal microscope imaging of a full-size auricle seeded with GFP-labeled cells, indicating the homogeneity of cell seeding within the construct. FIG. 4c, the two auricle designs, PCL scaffolds with and without the strengthening regions. FIG. 4d, stress-strain curve of the two areas (with and without the printed grid). FIG. 4e, the measured slope of the graph between the two lines indicating the difference in stiffness of the parts.

In some embodiments, to assess the porosity of the scaffold, the printed constructs were analyzed using SEM. In some embodiments, the scanning revealed large pores, approximately 500 μm wide, resulting from the grid mold/pore pattern and the small pores, ranging from 10-100 μm, resulting from the freeze-drying process (FIG. 4a). To ensure the seeded cells are evenly distributed within the PCL construct, GFP-expressing cells were used and imaged by a confocal microscope five days post-seeding; imaging revealed homogenous cell layers in all the dimensions (FIG. 4b).

In some embodiments, when designing a scaffold for auricle reconstruction, scaffold porosity should be ensured in order to allow cell attachment and matrix production, however, without lacking the mechanical stability necessary for the surrounding stress upon implantation. For this reason, strengthening regions (stiff zones) were added to the auricle design (FIG. 4c). In some embodiments, this was done by printing the BVOH molds having lower densities (or no density at all—meaning void) in specific areas, which will further allow polymer accumulation within them. In some embodiments, a rheometer test was performed to demonstrate the difference in the stiffness within the two regions (FIG. 4d). In some embodiments, the stress-strain curve revealed a significant difference between the two groups, on which the denser PCL was significantly stiffer (FIG. 4e).

### Cell Isolation and Expansion

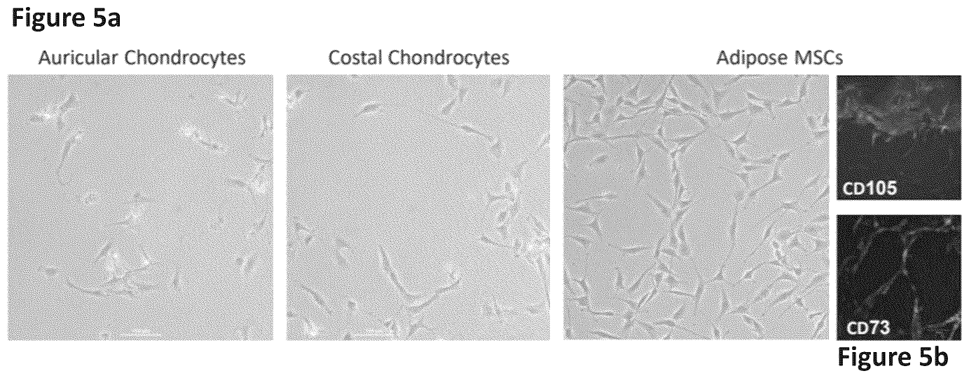

Referring now to FIGS. 5a-b showing: FIG. 5a, bright-field images of adipose-derived MSCs, Auricular, and rib chondrocytes at P0. scale bar=100 um; FIG. 5b, CD105 and CD73 immunofluorescent imaging of adipose-derived MSCs. scale bar=100 um.

In some embodiments, imaging of isolated chondrocytes from microtia tissue, costal tissue, and adipose-derived MSCs demonstrated viable and proliferative 2D-adherent cultures exhibiting typical morphology (FIG. 5a). In some embodiments, isolated MSCs purification was verified by immunofluorescent staining for CD105 and CD73 (FIG. 5b).

### Characterization of In Vitro Scaffold-Free Chondrogenic Differentiation Potential

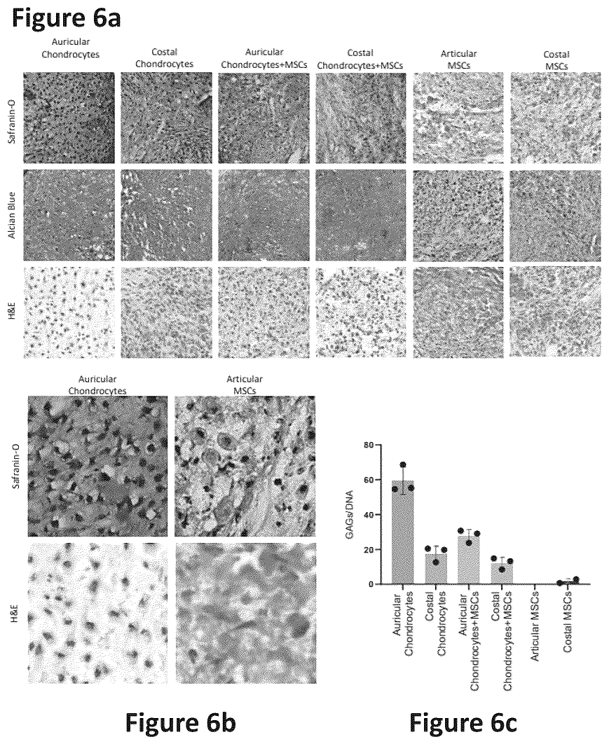

Referring to FIGS. 6a-c showing: FIG. 6a, safranin-O, alcian-blue, and H&E staining of sections of scaffold-free cartilage plugs composed of different combinations of cell types that were cultured for six-weeks in vitro. FIG. 6b, higher magnification of auricular chondrocyte-plug (right) versus adipose-derived MSCs-plug (left) safranin-O and H&E staining. FIG. 6c, DMMB biochemical assay was applied to the papain digested cartilage plugs.

In some embodiments, to analyze cells chondrogenic potential and the effect of the addition of MSCs to the culture, cartilage plugs were created from different cell combinations: auricular chondrocytes, costal chondrocytes, auricular adipose-derived MSCs, costal adipose-derived MSCs, and a co-culture of auricular chondrocytes with auricular adipose-derived MSCs and costal chondrocytes with costal adipose-derived MSCs. In some embodiments, cells were centrifuged to create plugs and were cultured for six weeks to allow differentiation and cartilage formation. In some embodiments, the plugs were then analyzed for initiation of cartilage formation; safranin-O, alcian-blue and hematoxylin and eosin staining (FIG. 6a) showed typical lacunas formation and secretion of cartilage components, collagen, and glycosaminoglycans within both auricular and costal chondrocytes. However, in cultured MSCs, safranin-O and lacunas appearance was absent (FIG. 6b). Furthermore, a quantitatively Dimethylmethylene Blue (DMMB) GAG assay indicate that cartilage secretion from auricular chondrocytes was higher compared to the other groups (FIG. 6c).

### Characterization of In Vitro Scaffold-Based Chondrogenic Differentiation Potential

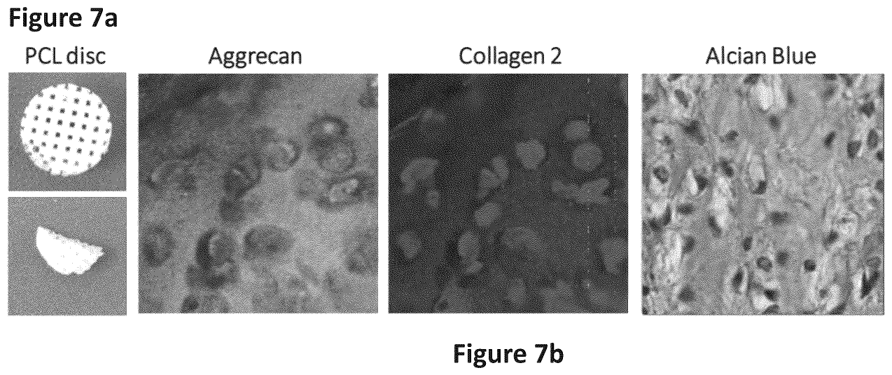

Referring to FIGS. 7a-b showing: FIG. 7a, PCL disc-shaped scaffolds were seeded with auricular chondrocytes and cultured for six-weeks followed by FIG. 7b, staining for Aggrecan, Collagen 2, Safranin-o, and alcian-blue cartilage markers.

In some embodiments, next, cartilage secretion from cells seeded in small PCL discs was examined (FIG. 7a), to enable the assessment of cartilage secretion under the same conditions as the auricle PCL scaffold. In some embodiments, chondrocytes were mixed with fibrin solution (Evicel, J&J) and seeded into 6 mm PCL disc scaffolds. In some embodiments, constructs were cultured for six-weeks allowing cells attachment, growth, migration, differentiation, and cartilage secretion within the discs. In some embodiments, whole-mount and sections staining of the constructs for aggrecan, collagen 2, and alcian-blue revealed the formation of lacunae (FIG. 7b).

### In Vivo Transplantation/Implantation of Human Auricle Grafts in a Murine Model

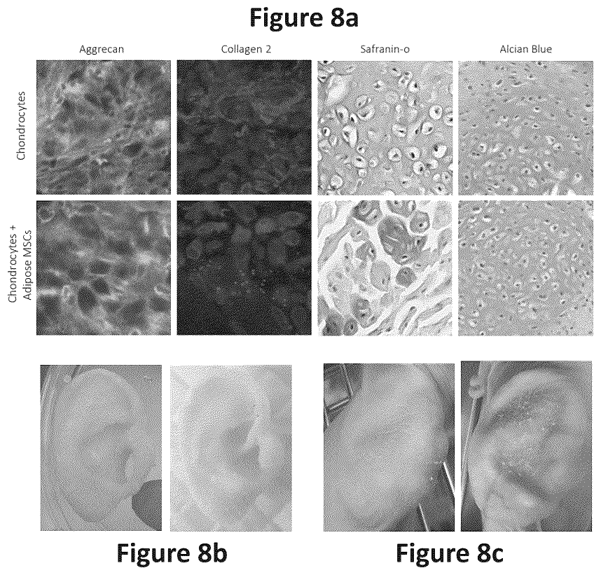

Referring to FIGS. 8a-c showing: FIG. 8a, PCL disc-shaped scaffolds were seeded with auricular chondrocytes with and without MSCs for six weeks and implanted subcutaneously within mice and then stained for Aggrecan, Collagen 2, Safranin-o, and alcian-blue cartilage markers. FIG. 8b, chondro-PCL-auricles without (left) and with strengthening regions (right). FIG. 8c, Cultured auricles with and without the strengthening parts were implanted subcutaneously and imaged eight weeks post-implantation.

In some embodiments, cartilage formation was further examined upon implantation. In some embodiments, cultured chondro-PCL discs with and without MSCs were implanted into the subcutaneous space of athymic nude mice. In some embodiments, twelve weeks post-implantation, mice were sacrificed, followed by dissection of the graft area, wholemount staining for aggrecan, collagen-2, and sections staining for safranin-O and alcian-blue. Staining demonstrated mature lacuna formation within the constructs (FIG. 8a).

In some embodiments, next, auricle graft stability upon implantation was assessed. In some embodiments, the auricle scaffold with and without strengthening regions was seeded with chondrocytes and MSCs (FIG. 8b) and was implanted into the subcutaneous space of athymic nude mice for 12-weeks. In some embodiments, images of the implanted auricle demonstrate that the strengthening regions have an important role in maintaining graft shape (FIG. 8c).

### Exemplary Embodiments of the Invention

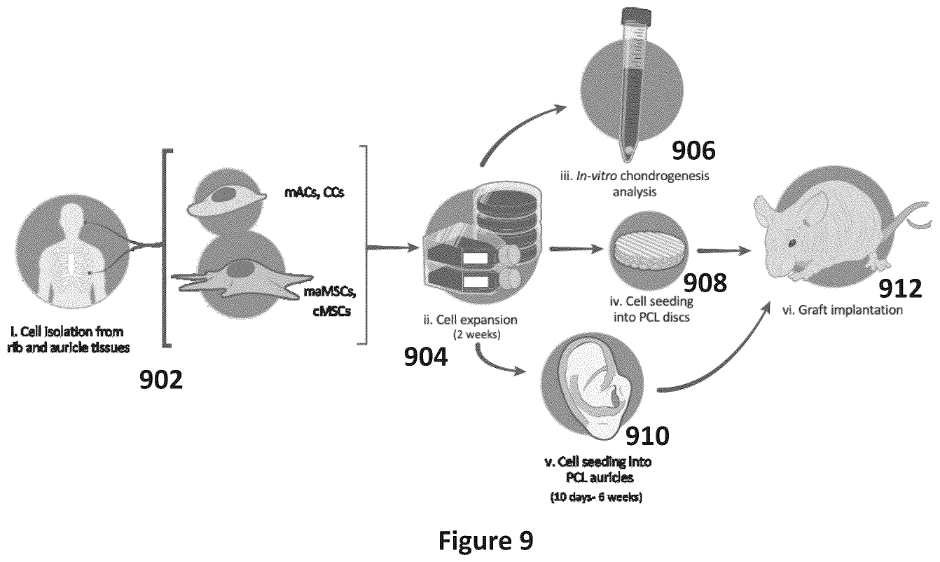

Referring now to FIG. 9, showing a schematic representation of an exemplary experimental design to prove the feasibility of the invention, according to some embodiments of the invention. The following experiments were performed to assess the feasibility of the invention, according to some embodiments of the invention.

In some embodiments, to bioengineer patient-specific ear-shaped cartilage for microtia patients, microtia auricular chondrocytes (mACs), costal chondrocytes (CCs), microtia auricular adipose-derived mesenchymal stem cells (maMSCs), and costal adipose-derived mesenchymal stem cells (cMSCs) are isolated 902 and expanded 904. In some embodiments, the chondrogenic potential of the cells is assessed using an in vitro plug assay 906. In some embodiments, molds were 3D-printed and filled with clinical-grade polycaprolactone (PCL) discs 908 and CT scanned-based PCL auricle constructs with strategically reinforced areas 910. In some embodiments, in vivo cartilage formation and stabilization post-implantation of the constructs are observed in a subcutaneous murine model 912.

In some embodiments, the scaffold preparation is performed, for example, as disclosed above and/or, for example, as disclosed in FIG. 3 for auricle scaffold preparation, and/or, for example as schematically shown in FIG. 10 for disc scaffold preparation. Referring now to FIG. 10, showing a schematic representation of an exemplary method of preparation of disc scaffold, according to some embodiments of the invention. In some embodiments, For example, PCL discs scaffold are molded using 3D printing and freeze-drying. In some embodiments, the discs are created by printing Butenediol Vinyl Alcohol Co-polymer (BVOH) meshed molds 1002. In some embodiments, then, 600 μl medical-grade PCL solution is poured into the mold 1004, followed by rapid construct freezing to avoid evaporation 1006. In some embodiments, samples are then lyophilized to form pores within the polymer bulk, and then washed with water to remove the BVOH mold 1008. In some embodiments, the final scaffold is dried and sterilized 1010.

In some embodiments, exemplary cell isolation methods are as disclosed above. Alternatively or additionally, for example, as following:

Specifically, the cells used in the experiment were collected from 4 different patients (age 10-12 years, male and female). In some embodiments, cartilage remnants are taken from the microtic auricle, rib cartilage and adipose tissue remnants after sculpturing the new auricle (See Table 1 below).

In some embodiments, as mentioned above, to isolate the chondrocytes, either rib cartilage or auricular cartilage are cut into small pieces (approximately 1 mm), followed by a PBS with 1% penicillin-streptomycin (pen-strep, biological-industries) wash and incubation with collagenase 2 for 12-14 hours. In some embodiments, the sample is then passed through a 100 μm strainer. In some embodiments, growth medium (40 ml DMEM F12 with 10% fetal bovine serum (FBS) and 1% Penstrep) is added to the filtered sample, which is then centrifuged (2000 g, 10 min). In some embodiments, the medium is aspirated, and the cells are resuspended in a growth medium and seeded at a density of 5000 cells/mm².

In some embodiments, as mentioned above, to isolate the adipose-derived MSCs, either rib fat or auricular fat are cut into small pieces and incubated with 2 mg/ml collagenase 1 in DMEM (10 ml collagenase for every 3 g tissue) and incubated for 1 h, 37° C. In some embodiments, DMEM F12 (Biological Industries) with 10% FBS is added to the solution in equal parts to the collagenase 1, and the sample is then filtrated through a 100 μm and then a 40 μm strainer. In some embodiments, the solution is centrifuged for 7 min, 500 g. In some embodiments, the medium is aspirated, and the cells are resuspended with the growth medium. In some embodiments, one day later, the medium is replaced with Nutristem (Biological Industries) and exchanged every 2-3 days.

TABLE 1
A listing of all isolated cells

| Cell type | Acronym |
|---|---|
| Microtia auricular chondrocytes | mACs |
| Costal chondrocytes | CCs |
| Microtia auricular adipose-derived mesenchymal stem cells | maMSCs |
| Costal adipose-derived mesenchymal stem cells | cMSCs |

### Exemplary Characterization Using Flow Cytometry

In some embodiments, flow cytometry is used to characterize mesenchymal phenotype using a mesenchymal stem cell marker antibody panel (R&D Systems). In some embodiments, maMSCs are fixed in 4% PFA (ChemCruz, Santa Cruz) for 10 min at 4° C. In some embodiments, to remove the fixation solution, the cells are centrifuged at room temperature (RT) for 2 min at 600 g and then washed in 2% FBS (Gibco) in PBS 1× (Sigma). In some embodiments, the cells are incubated with the primary antibodies for 30 min at RT and washed in 2% FBS in PBS 1× and centrifuged at RT for 4 min at 300 g. In some embodiments, next, the cells are incubated with a secondary antibody, for example, donkey anti-mouse Alx647 (Jackson, 1:400), for 30 min at RT in the dark. In some embodiments, the cells are washed in 2% FBS in PBS 1× and centrifuged at RT for 4 min at 300 g. In some embodiments, cells are analyzed using, for example, a LSR-II flow cytometer (BD) instrument and the data is assessed, for example, with FlOWJO (BD) Analysis software.

### Exemplary Cell Seeding

In some embodiments, as mentioned above, before seeding, PCL discs and/or auricles are washed three times with PBS and then with a growth medium to create a hydrophilic environment for cell seeding. In some embodiments, the construct is then stained with phenol red.

In some embodiments, chondrocytes and MSCs are trypsinized using 2× trypsin (Gibco) and resuspended with 7.5 U thrombin (EVICEL). In some embodiments, a BAC2 solution (15 mg/ml; the same volume as the thrombin) is then added to the cells, which were then seeded onto the auricle or scaffold. In some embodiments, unloaded areas are easily detected as they remained red, whilst the phenol red color disappeared from the seeded areas. In some embodiments, cells are seeded at a density of 50×10⁶ cells/ml along with fibrin to mediate cell attachment. In some embodiments, for example, for the auricle, 800 μl (40×10⁶ cells) of the fibrin solution is used. In some embodiments, for example, for the PCL discs, 20 μl (875×10³ cells) of fibrin are used.

In some embodiments, seeded constructs are incubated (1 h, 37° C.), after which a growth medium is added. In some embodiments, two days after seeding, differentiation medium (DMEM F12 (pen-strep (1%, Biological Industries), TGF-□ (10 ng/ml, Prospec), ITS premix (50 mg/ml, Corning), ascorbic acid (50 μg/ml, Sigma), dexamethasone (100 nM, Sigma), amphotericin B (0.25 μg/ml, Biological Industries)) is added. In some embodiments, the volume of medium added depended on its size; for example, 150 ml for the 100% size auricle, 100 ml for the 70% size auricle and 4 ml for the discs.

In some embodiments, to demonstrate homogenous cell seeding and spreading, GFP expressing fibroblasts (Angioproteomie) are seeded into PCL auricle scaffolds in the same manner as described above. In some embodiments, constructs were imaged 5 days post-seeding using a confocal microscope (LSM700, Zeiss).

### Exemplary Chondrocyte Plugs

In some embodiments, as mentioned above, different combinations of chondrocytes (auricular or rib origin) and adipose-derived MSCs (auricular or rib origin) are mixed and centrifuged for 5 min at 400 g to create plugs. In some embodiments, for the co-culture group, MSCs and chondrocytes were mixed in a 1:1 ratio. In some embodiments, basal medium is added to the plugs for the first two days, after which differentiation medium is added. In some embodiments, the medium is changed every 2-3 days. In some embodiments, after 6 weeks of culture, the plugs are fixed with 4% Paraformaldehyde for 10 minutes, washed 3 times with PBS, incubated in 30% (wt/vol) sucrose solution, embedded in optimal cutting temperature compound (Tissue-Tek) and frozen for subsequent cryosectioning (5-7 μm). 3 plugs were created per group.

### Exemplary Graft Implantation

In some embodiments, as mentioned above, athymic nude mice (male, 7-9-weeks-old; Harlan Laboratories) are anesthetized with isofluorane. In some embodiments, small (˜2 cm) and large (˜4 cm) incisions are made in the skin for the discs and auricles, respectively, and grafts are transplanted in the subcutaneous space. In some embodiments, the skin is then sutured with 5-0 absorbable sutures. In some embodiments, mice are sacrificed after 12 weeks, followed by excision of the grafts. In some embodiments, the grafts are then cut into 3 pieces for mechanical testing, staining, and biochemical analysis.

### Exemplary Number of Constructs Used in the Animal Experiments:

For the different cell combination assessment: mAC—3 constructs, CCs—6 constructs, CCs+cMSCs—3 constructs, mACs+CCs—3 constructs, mACs+maMSCs—2 constructs.

For short vs. long in-vitro incubation time: for the 10 days—4 constructs in 4 mice and for the 6 weeks—2 constructs in 2 mice.

For the full ear experiments: 3 with reinforced regions and 3 without reinforced regions.

### Exemplary Immunofluorescent and Biochemical Staining

In some embodiments, constructs are dehydrated in ethanol and embedded in paraffin at 65° C. In some embodiments, sections (5 μm-thick) are cut, deparaffinized with xylene and rehydrated, then stained with Alcian-blue, safranin-O with fast green and hematoxylin and eosin.

In some embodiments, for immunofluorescent staining, sections are incubated in chondroitinase ABC (0.25 U/ml, Sigma-Aldrich) in tris-acetate buffer (1×, Sigma-Aldrich) (1 h, 37° C.) and then in keratinase (0.25 U/ml, Sigma-Aldrich) in tris-acetate buffer (1×, Sigma-Aldrich; 30 min, 37° C.). In some embodiments, constructs are then washed with PBS and immersed overnight in BSA solution (5%; Millipore).

In some embodiments, samples are then incubated (overnight, 4° C.) with anti-human collagen 2 (1:250; R&D), anti-human aggrecan (1:500; R&D) and anti-human elastin (1:1000, Sigma-Aldrich) antibodies. In some embodiments, after extensive rinsing, they are then incubated (3 h, room temperature) with Cy3-labeled (1:100; Jackson Immunoresearch Laboratory), Alexa-488 (1:400; ThermoFisher Scientific), and Alexa-647 (1:400; ThermoFisher Scientific) secondary antibodies mixed with DAPI (Sigma-Aldrich).

In some embodiments, for 2D plate immunofluorescent staining, cells are fixated in paraformaldehyde (4%) for 20 min and then permeabilized with 0.3% Triton X-100 (Bio Lab Ltd.) for 10 min. In some embodiments, constructs are then washed with PBS and immersed in BSA solution (5%; Millipore) overnight. In some embodiments, samples are then incubated with the following primary antibodies for 2 h: mouse anti-human CD73 (1:100; Acris), mouse anti-human 105 (1:100; BD). In some embodiments, cells are then treated with Alexa-488-labeled (1:400; ThermoFisher Scientific) secondary antibodies and DAPI (Sigma-Aldrich), for 2 h, at room temperature.

### Exemplary Biochemical Assays

In some embodiments, as mentioned above, in vitro and in vivo samples are digested with papain solution (40 μg/ml diluted in 20 nM ammonium acetate, 1 mM EDTA, and 2 mM dithiothreitol) for 48 h, at 65° C. In some embodiments, DNA content is measured using the Hoechst dye-binding assay. In some embodiments, proteoglycan concentration is quantified by measuring the amount of sulfated GAG using the 1,9-dimethylmethylene blue (DMMB) dye-binding spectrophotometric assay. In some embodiments, collagen content is quantified by adding 6N hydrochloric acid for 18 h, 110° C. and neutralization with NaOH. In some embodiments, hydroxyproline level is measured using the chloramine-T/Ehrlich's spectrophotometric assay. In some embodiments, the experiments are done with at least two technical replicates for each sample.

Exemplary microCT Imaging

In some embodiments, to further assess the macro-structure of the construct, PCL constructs are scanned using a high-resolution microCT scanner (Skyscan 1276, Bruker, Kontich, Belgium), using the following exemplary scanning parameters: source voltage 65 kV, source current 57 μA, applied 0.5 mm aluminum filter. In some embodiments, scanning is performed using a 0.3-degree rotation step with frame averaging (3), yielding a total of 1201 projections. In some embodiments, image acquisition is performed with a scaled pixel size of 21 μm. In some embodiments, back projections were reconstructed using NRecon (Skyscan, version 1.7.2.0), CTAnn Software (Skyscan, version 1.17.7.2) is used for segmentation, and CTVox (Skyscan, version 2.2.0) was used for 3D visualization.

### Exemplary Confocal Imaging

In some embodiments, constructs sections with immunofluorescent staining are imaged using a confocal microscope (LSM700; Zeiss), with 2.5×, 5×, 10×, 20× and 40× lenses. In some embodiments, for biochemical staining, sections are imaged using an inverted microscope (Axio Observer; Zeiss), with 5×, 10×, 20× and 40× lenses. In some embodiments, the samples were imaged in at least three regions and representative images are presented.

### Exemplary Mechanical Testing

In some embodiments, to determine the stiffness of each region, two types of 6 mm disc molds are fabricated and filled with PCL solution; in some embodiments, the first is with no infill to ensure the same structure as the reinforced region and, in some embodiments, the second is with 50% rectilinear infill to mimic the structure of the auricle bulk.

In some embodiments, stress-strain curves are generated using an AR-G2 rheometer (TA Instruments, New Castle, DE, USA) equipped with parallel-plate geometry. In some embodiments, samples are compressed to 90% of their original width, and stress was then calculated by dividing the measured force by the cross-section area. In some embodiments, Young's modulus is determined by calculating the slope of the stress-strain curve in the linear region. In some embodiments, for in-vivo auricle samples, samples are cut into ˜20 mm² pieces before testing and imaged immediately after testing to find the cross-section area.

### Exemplary Statistical Analysis

In some embodiments, presented data include the mean±standard deviation. In some embodiments, Student's t-test is performed to compare two groups. In some embodiments, to examine differences between multiple groups, a one-way analysis of variance (ANOVA) is performed, with posthoc Tukey's multiple comparisons. In some embodiments, results are considered significant for p<0.05. In some embodiments, statistical analysis is performed using GraphPad Software, a computerized statistical program.

### Exemplary Results of the Experiments

### Characterization of the Chondrogenic Potential of Isolated Cells

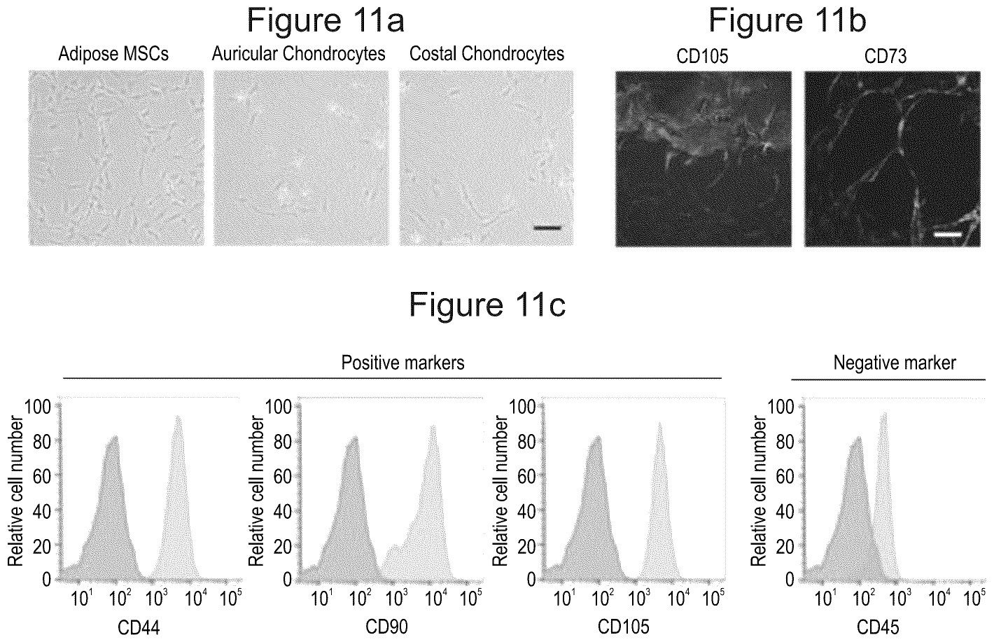

Imaging of microtia auricular chondrocytes (mAC), costal chondrocytes (CCs), and adipose-derived MSCs demonstrated viable and proliferative 2D-adherent cultures exhibiting typical morphology (FIG. 11a). The purity of the isolated MSCs was verified by immunofluorescence staining for CD105 and CD73 (FIG. 11b). Moreover, the immunophenotype of the MSCs was analyzed using flow cytometry which demonstrated positive expression of mesenchymal markers CD44, CD90, CD105 and low expression of the hematopoietic marker CD45 (FIG. 11c).

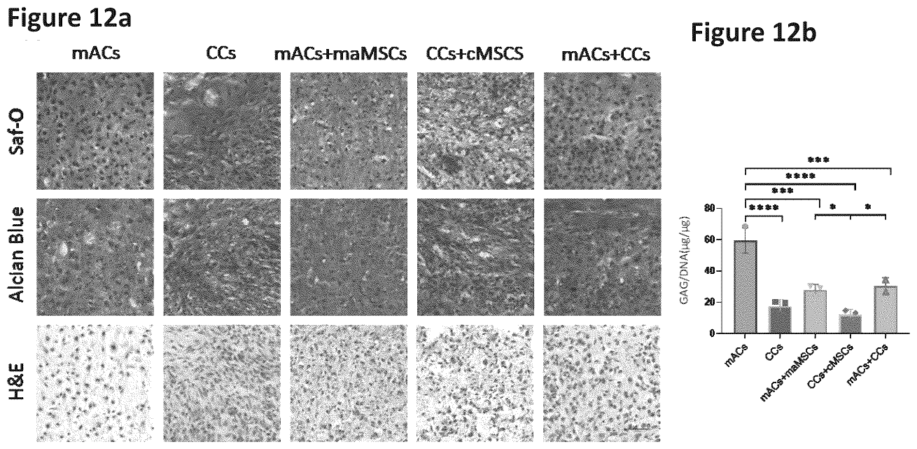

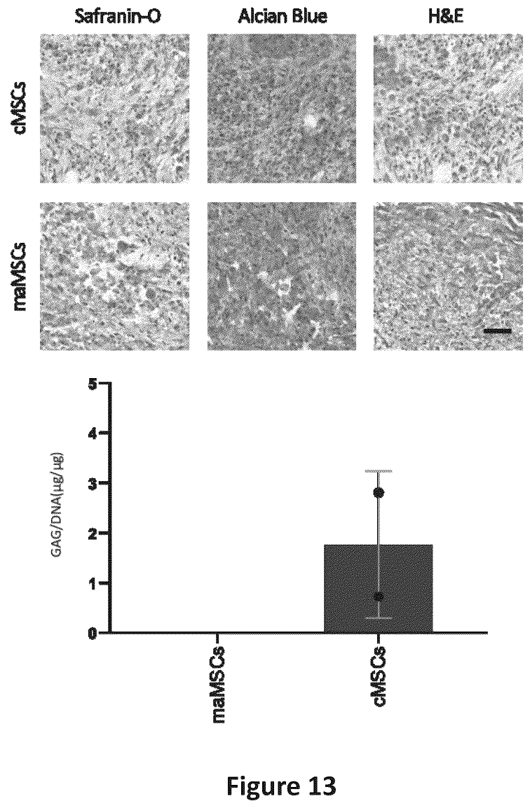

Chondrogenic potential and the effect of the addition of MSCs on cultured chondrocytes were assessed in cartilage plugs comprised of monoculture of mACs, CCs, microtia maMSCs and cMSCs. In addition, to examine additional cell combinations, three co-culture groups were seeded: mACs with maMSCs, CCs with cMSCs, and mACs with CCs. Plugs prepared with both auricular and costal chondrocytes were analyzed for cartilage formation initiation; safranin-O, alcian-blue and hematoxylin and eosin staining showed typical lacunae formation and secretion of glycosaminoglycans. Safranin-O and alcian-blue staining revealed no differences between the different groups, with the exception of MSCs plugs, for which safranin-O staining was absent and alcian blue staining was pale (FIG. 12a, FIG. 13). GAGs normalized to DNA content revealed that cartilage formation from mAC plugs was higher than any other groups, CCs, mACs+maMSCs and mACs+CCs produced fewer GAGs per cells but still had more than CCs+cMSCs (FIG. 12b).

### Characterization of Scaffold-Based Chondrogenic Potential

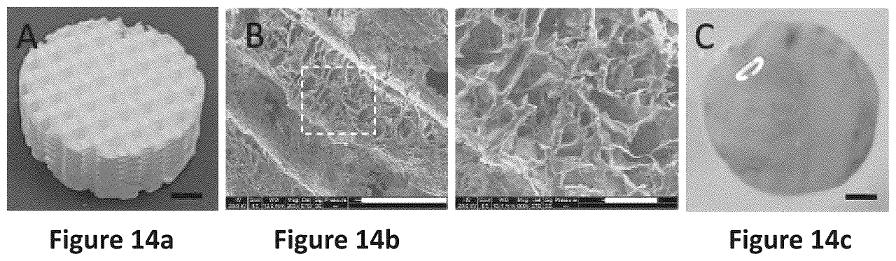

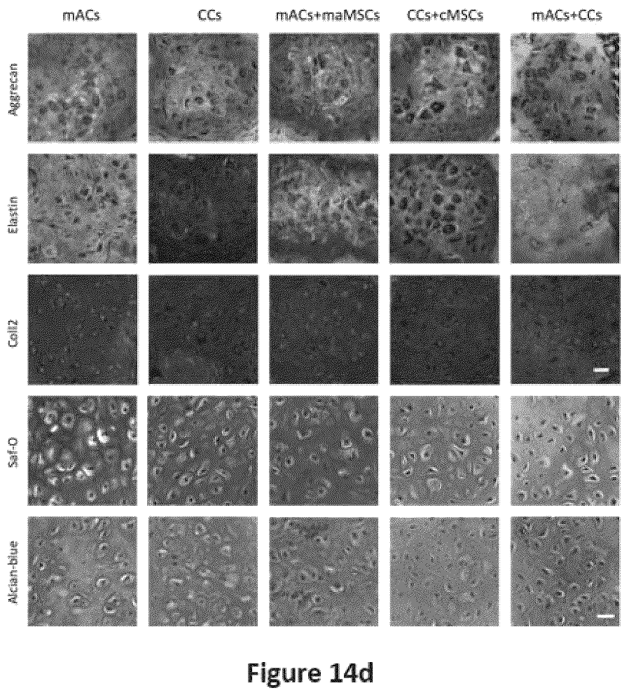

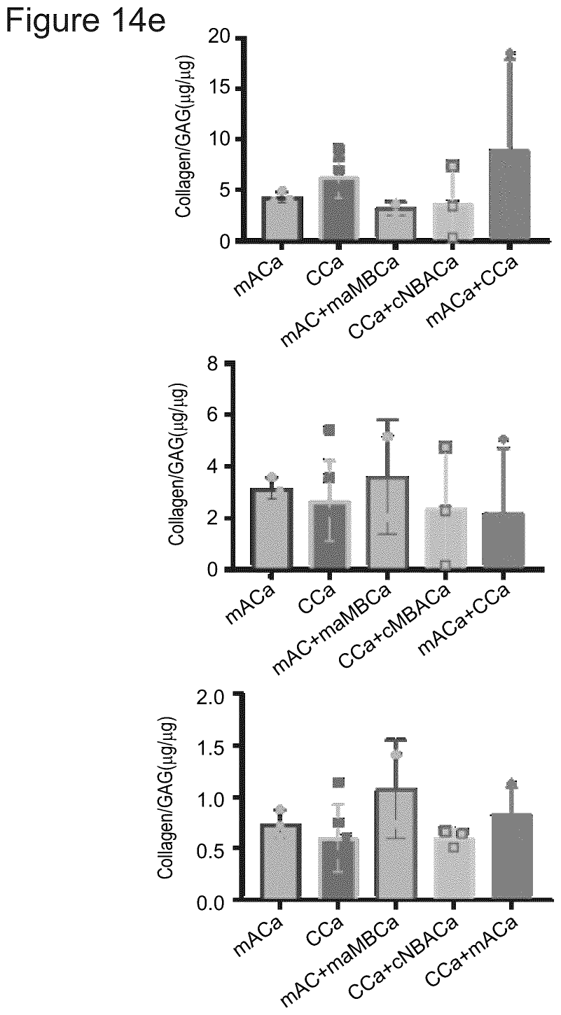

Small PCL discs were designed to enable the assessment of cartilage formation under the same conditions as large auricle PCL scaffolds. The use of small discs is simpler to produce and test and can aid in determining the optimal conditions for auricle construct fabrication. The resulting scaffold contained large pores (˜500 μm), formed from the printed mold, and small pores, formed during the freeze-drying process (˜100 um) (FIGS. 14a and 14b).

Different cell combinations of mACs, CCs, ACs with maMSCs, CCs with cMSCs, mACs with CCs were mixed with fibrin solution and seeded into 6 mm PCL disc scaffolds, cultured in vitro for 6 weeks, and implanted into the subcutaneous space of athymic nude mice. Mice were sacrificed 12 weeks post-implantation, followed by dissection of the graft area (FIG. 14c). Staining of the constructs extracted 12 weeks post-implantation revealed lacunae formation within all groups (FIG. 14e). No significant differences in GAGs and collagen secretion were observed between the different cell combinations (FIG. 14e). Therefore, other cell combinations can be integrated into the construct when needed.

### In Vitro Culture Time Effect on Cartilage Formation

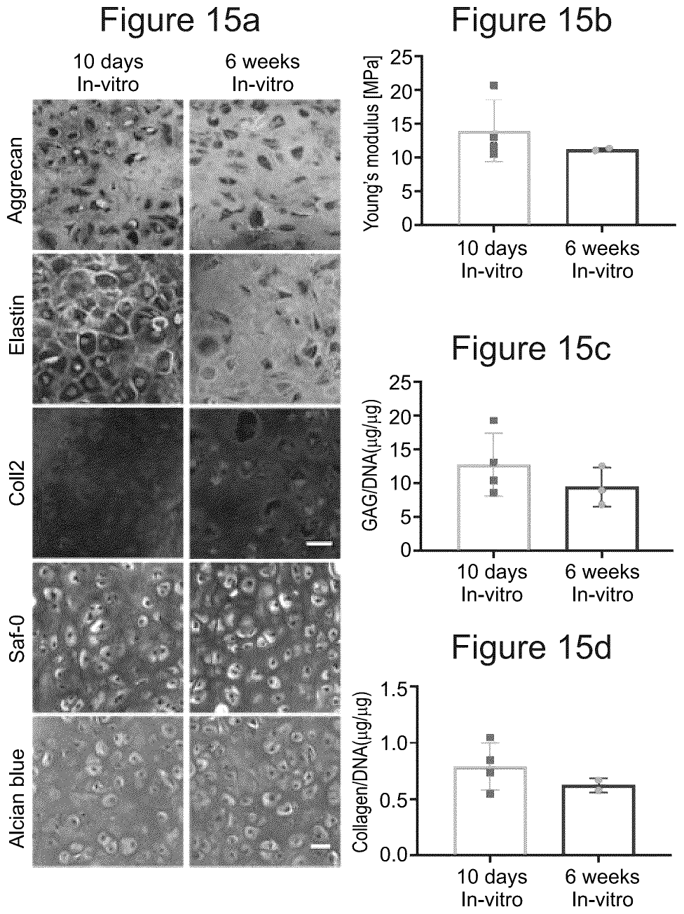

Due to the logistic and technical benefits of shortening the in vitro culture time, including reduced time to implantation, reduced culturing costs and reduced possibility of graft contamination, the impact of in vitro culture time on cartilage formation was assessed for two different mACs grafts. Grafts cultured for 10 days or for 6 weeks prior to implantation exhibited similar morphology, with typical lacunae formation (FIG. 15a). In addition, the two sets of grafts showed similar Young's modulus (FIG. 15b). No significant differences in GAGs and collagen levels of the two implant groups (FIG. 15c-15d). Taken together, a short in vitro incubation time is sufficient to achieve cartilage production.

### Ear-Shaped Scaffold Characterization

When designing a scaffold for auricle reconstruction, scaffold porosity must be sufficient to enable cell attachment and matrix production (FIG. 4c) while being sufficiently mechanically stable to withstand the surrounding stress upon implantation. To ensure these traits, reinforced regions were introduced into the auricle design (FIG. 4c, bottom left) by printing the BVOH molds at lower densities in specific areas, which would enable increased polymer accumulation within them. The resulting auricle was subsequently composed of two areas—the supporting frame with a highly dense polymer with small pores ˜100 μm, formed during the freeze-drying process (FIG. 4a or FIG. 14b) and the bulk area with pores formed during both the freeze-drying process and by the BVOH mold. In this manner, the auricle scaffold shape was close to the native auricle, with reinforced regions to withstand pressure upon implantation and varying interconnected pores to allow cell spreading, migration and ECM secretion (FIG. 4c, bottom right).

Rheology testing was performed to demonstrate the difference in stiffness between the two regions (FIGS. 4d, 4e). A stress-strain analysis identified a different mechanical response of the two areas to compression. Slope measurement of the first linear range revealed a significant difference in Young's modulus between the areas of the construct with higher (˜2 MPa) versus lower PCL density (˜0.2 MPa) (FIG. 4e).

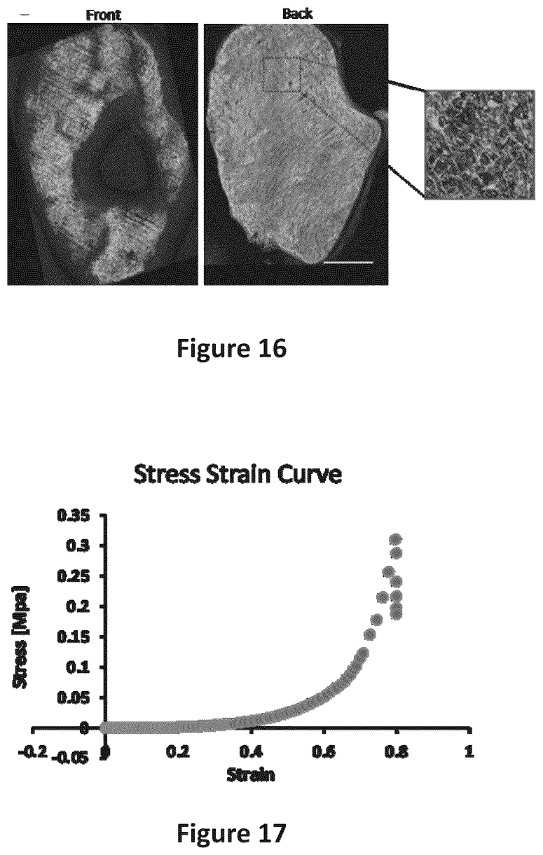

To ensure the seeding process resulted in an even distribution of the cells, GFP-expressing cells were seeded on the PCL auricle constructs; confocal images revealed that the cells formed homogenous layers in all dimensions of the constructs (FIG. 16).

### In Vivo Implantation of Human Auricle Grafts in a Murine Model

After validating the chondrogenic potential of embedded cells and designing the appropriate auricle scaffold, the mechanical and morphological stability of mAC-embedded auricle grafts were assessed 12 weeks after their subcutaneous implantation into athymic nude mice. Images of the implanted auricle demonstrated that the reinforced regions played a crucial role in maintaining graft shape; all curvatures and details were maintained, whereas without the reinforced regions, the auricle deformed and did not retain its original shape (FIG. 8c). The constructs' Young's modulus value extracted from the stress strain curve (FIG. 17), 2.55±0.793 MPa, was similar to that of a native auricle, 1.73 MPa. The samples contained lacunae and typical cartilage ECM secretion (FIG. 8a).

As used herein with reference to quantity or value, the term “about” means “within ±20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A medical grade implant, comprising:
a. a biodegradable scaffold;
b. a plurality of seeded cells on the surface of said scaffold;
wherein said biodegradable scaffold comprises parts with different levels of stiffness.

2. The medical grade implant according to claim 1, wherein said parts with different levels of stiffness comprise different quantities of scaffold material.

3. The medical grade implant according to claim 1, wherein parts having high levels of stiffness comprise one or more of:
a. more scaffold material; and
b. less openings or no openings at all.

4. The medical grade implant according to claim 1, wherein parts having low levels of stiffness comprise one or more of:
a. less scaffold material; and
b. openings.

5-6. (canceled)

7. The medical grade implant according to claim 4, wherein said low levels of stiffness are from about 1.5 MPa to about 5 MPa.

8. (canceled)

9. The medical grade implant according to claim 3, wherein said high levels of stiffness are from about 15 MPa to about 30 MPa.

10. (canceled)

11. The medical grade implant according to claim 1, wherein said scaffold comprises an ultimate tensile strength of from about 10% PCL/0.25 MPa to 30 30% PCL/2 MPa.

12. The medical grade implant according to claim 1, wherein said parts that comprise more scaffold material comprise from about 5% to about 80% more scaffold material than parts comprising less scaffold material.

13. (canceled)

14. The medical grade implant according to claim 1, wherein said implant allows cell growth and regeneration of cartilage tissue at the implant site.

15. A method of manufacturing a 3D printed implant, comprising:
a. printing a mold of a scaffold;
b. generating said scaffold from said mold;
c. seeding cells on said scaffold;
wherein said generating said scaffold comprises generating parts of said scaffold with different levels of stiffness.

16. The method according to claim 15, further comprising one or more of:
a. virtually planning said scaffold using a scanned image of an anatomical structure of a subject in need of said 3D printed implant; and
b. virtually generating a mold for said planned scaffold to be used as instructions for said printing.

17. (canceled)

18. The method according to claim 15, wherein said printing comprises using at least one printing material for said printing.

19. The method according to claim 18, wherein said generating parts with different levels of stiffness comprises printing parts of said mold with more of said at least one printing material than other parts in said mold; and
wherein parts comprising more of said at least one printing material in said mold provide stiffer parts in said scaffold during said generating.

20. (canceled)

21. The method according to claim 18, wherein parts comprising less of said at least one printing material comprise openings;
wherein in said parts comprising openings less scaffold material is retained during said generating of said scaffold from said mold; and
wherein less retained scaffold material provides parts with lower levels of stiffness in said scaffold.

22-23. (canceled)

24. The method according to claim 15, wherein parts comprising more printing material comprise less openings or no openings at all;
wherein in said parts comprising less openings or no openings at all more scaffold material is retained during said generating of said scaffold from said mold; and
wherein more retained scaffold material provides parts with higher levels of stiffness in said scaffold.

25-26. (canceled)

27. The method according to claim 15, wherein lower levels of stiffness are from about 1.5 MPa to about 5 MPa; and
wherein higher levels of stiffness are from about 15 MPa to about 30 MPa.

28-30. (canceled)

31. The method according to claim 15, wherein said scaffold comprises an ultimate tensile strength of from about 10% polycaprolactone (PCL)/0.25 MPa to about 30% PCL/2 MPa.

32. The method according to claim 15, wherein said parts that comprise more scaffold material comprise from about 5% to about 80% more scaffold material than parts comprising less scaffold material.

33. (canceled)

34. The method according to claim 15, wherein said implant allows cell growth and regeneration of cartilage tissue at the implant site.

35. A method for producing a custom implant for cartilage repair, comprising:
a. manufacturing a three-dimensional mesh mold using a scanned image of an anatomical structure of a subject in need; wherein said manufactured three-dimensional mesh mold serves as a supporting scaffold for said custom implant;
b. generating said scaffold from said mold;
c. seeding cells on said scaffold;
wherein said generating said scaffold comprises generating reinforcement zones in said scaffold at predefined areas that allow the structural stability of said implant after implantation; further wherein said implant allows cell growth and the regeneration of cartilage tissue at the implant site.
